(19) United States
(12) Patent Application Publication
(10) Pub. No.: US 2024/0347216 A1
(43) Pub. Date: Oct. 17, 2024

(54) INFORMATION PROCESSING SYSTEM, INFORMATION PROCESSING APPARATUS, INFORMATION PROCESSING METHOD, AND NON-TRANSITORY COMPUTER READABLE MEDIUM STORING PROGRAM

(71) Applicant: NEC Corporation, Minato-ku, Tokyo (JP)

(72) Inventors: Yoshifumi ONISHI, Tokyo (JP); Koichi NIHEI, Tokyo (JP); Takanori IWAI, Tokyo (JP)

(73) Assignee: NEC Corporation, Minato-ku, Tokyo (JP)

(21) Appl. No.: 18/682,517

(22) PCT Filed: Sep. 29, 2021
(86) PCT No.: PCT/JP2021/035857

§ 371 Date: Feb. 9, 2024

(51) Int. Cl. G16H 80/00; G16H 40/20; G16H 40/67

(52) U.S. Cl. CPC G16H 80/00; G16H 40/20; G16H 40/67

(57) ABSTRACT

An information processing system (100) according to the present disclosure includes communication status acquisition means (101) for acquiring a communication status, the communication status being one when a first terminal receives a video image including a subject from a second terminal through a network, and instruction means (102) for outputting instruction information for guiding at least one of a user of the first terminal, a user of the second terminal, and the subject to at least one of the first and second terminals based on the) acquired communication status. Further, the first terminal includes first display means for displaying a video image of the subject and displaying the instruction information from the instruction means, and the second terminal includes second display means for displaying the instruction information from the instruction means.

### TECHNICAL FIELD

The present disclosure relates to an information processing system, an information processing apparatus, an information processing method, and a non-transitory computer readable medium storing a program.

### BACKGROUND ART

Patent Literature 1 discloses a remote diagnosis system in which a communication terminal apparatus, a server apparatus, and a medical institution terminal are connected to each other through a network. Image data obtained by photographing a diseased part of a patient by a camera provided in the communication terminal apparatus, inquiry data, and position data are transmitted to the server apparatus through the network. In this remote diagnosis system, when a remote diagnosis is carried out, in addition to the health condition of the patient, local information on the remote area where the patient is currently present is acquired, and diseases unique to the local area and diseases currently prevalent in that area are taken into consideration.

Patent Literature 2 discloses an ultrasonic diagnostic apparatus that measures characteristics of a biologic tissue, such as a shape, a motion characteristic, and a property characteristic of the biologic tissue, converts them into video information, and transmits the obtained video information through a network. This ultrasonic diagnostic apparatus periodically monitors the communication status of the network and selects a video format for transmitting data or the like to an external apparatus according to the monitored communication status, and by doing so, it maintains the time resolution required for diagnoses.

### CITATION LIST

### Patent Literature

Patent Literature 1: Japanese Unexamined Patent Application Publication No. 2012-221447
Patent Literature 2: Japanese Unexamined Patent Application Publication No. 2007-190154

### SUMMARY OF INVENTION

### Technical Problem

In a medical service providing a system in which a doctor makes a diagnosis based on a video image of a patient, in some cases, biometric information is measured based on information indicating a change in the state of the body surface of the patient shown in the video image. If the communication status of the network deteriorates during such a measurement of biometric information, the reliability of the measured biometric information may deteriorate. Therefore, there is a concern that the quality of the medical service will deteriorate.

In Patent Literature 2, in order to adapt (i.e., adjust) the amount of video information to be transmitted to an amount corresponding to the tolerable transmission amount of the network, either an image processing method having a high image compression ratio or an image processing method having a low image compression ratio is selected as an appropriate video format according to the communication status of the network. However, when biometric information is measured from a video image of a patient, it is difficult to obtain reliable biometric information from video information on which some processing has been performed.

An object of the present disclosure is to provide an information processing system, an information processing apparatus, an information processing method, and a non-transitory computer readable medium storing a program capable of preventing the quality of a medical service from deteriorating due to deterioration or the like of a communication status.

### Solution to Problem

An information processing system according to an aspect includes: communication status acquisition means for acquiring a communication status, the communication status being one when a first terminal receives a video image including a subject from a second terminal through a network; and instruction means for outputting instruction information for guiding at least one of a user of the first terminal, a user of the second terminal, and the subject to at least one of the first and second terminals based on the acquired communication status.

An information processing apparatus according to an aspect includes: communication status acquisition means for acquiring a communication status, the communication status being one when a first terminal receives a video image including a subject from a second terminal through a network; and control means for performing control so as to output instruction information for guiding at least one of a user of the first terminal, a user of the second terminal, and the subject to at least one of the first and second terminals based on the acquired communication status.

An information processing method according to an aspect includes: acquiring a communication status, the communication status being one when a first terminal receives a video image including a subject from a second terminal through a network; and outputting instruction information for guiding at least one of a user of the first terminal, a user of the second terminal, and the subject to at least one of the first and second terminals based on the acquired communication status.

A non-transitory computer readable medium according to an aspect stores a program for causing a computer to perform processes including: acquiring a communication status, the communication status being one when a first terminal receives a video image including a subject from a second terminal through a network; and outputting instruction information for guiding at least one of a user of the first terminal, a user of the second terminal, and the subject to at least one of the first and second terminals based on the acquired communication status.

### Advantageous Effects of Invention

According to the above-described aspect, it is possible to provide an information processing system, an information processing apparatus, an information processing method, and a non-transitory computer readable medium storing a program capable of preventing the quality of a medical service from deteriorating due to deterioration or the like of a communication status.

### BRIEF DESCRIPTION OF DRAWINGS

FIG. 1 is a block diagram showing a configuration of an information processing system according to an example embodiment;

FIG. 2 shows an example of a configuration of an information processing system including an information processing apparatus according to an example embodiment;

FIG. 3 is a block diagram showing an example of a configuration of a doctor-side terminal shown in FIG. 2;

FIG. 4 is a block diagram showing an example of a configuration of a patient-side terminal shown in FIG. 2;

FIG. 5 is a diagram for explaining an example of contents displayed in the doctor-side terminal;

FIG. 6 is a diagram for explaining an example of contents displayed in the patient-side terminal; and

FIG. 7 is a sequence diagram for explaining an example of a flow of an information processing according to an example embodiment.

### EXAMPLE EMBODIMENT

An example embodiment according to the present disclosure will be described hereinafter with reference to the drawings. Note that the following descriptions and drawings have been omitted and simplified as appropriate for clarifying the explanation. Further, the same reference numerals (or symbols) are assigned to the same elements throughout the drawings, and redundant descriptions thereof are omitted as appropriate. Further, specific numerical values and the like shown below are merely examples for facilitating understanding of the present disclosure, and they are not limited to those shown below.

An example embodiment relates to a medical service providing technology in which video data of a subject is transmitted, through a network, between a facility in which medical services are provided and another facility which is remotely located from the aforementioned facility and in which a subject who receives a medical service is present, and an expert such as a doctor makes a diagnosis based on a video image(s) of the subject.

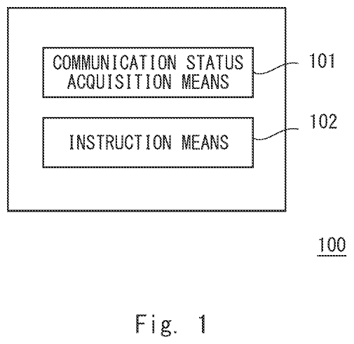

FIG. 1 is a block diagram showing a configuration of an information processing system 100 according to an example embodiment. As shown in FIG. 1, the information processing system 100 includes communication status acquisition means 101 and instruction means 102. The acquisition means 101 acquires a communication status which is one obtained (e.g., measured) when a first terminal receives a video image including a subject (i.e., containing an image of a subject) from a second terminal through a network. Based on the acquired communication status, the instruction means 102 outputs instruction information for guiding at least one of the user of the first terminal, the user of the second terminal, and the subject to at least one of the first and second terminals.

As described above, since instruction information for guiding at least one of the user of the first terminal, the user of the second terminal, and the subject is output while taking the communication situation into consideration, the person who has received the instruction information can take actions by referring to the instruction information. As the person who has received the instruction information takes actions as described above, it is possible to prevent the quality of the medical service from deteriorating due to deterioration or the like of the communication status.

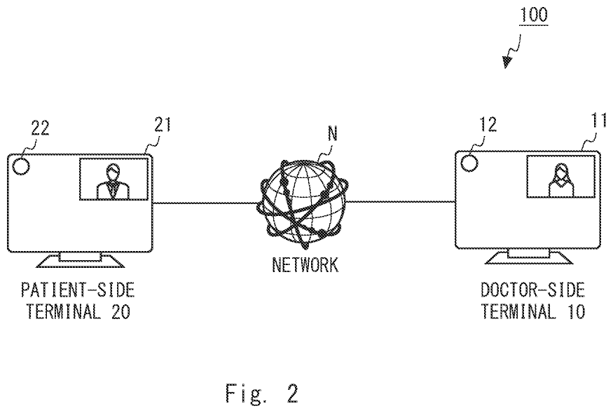

Next, a specific example of the information processing system 100 according to an example embodiment will be described with reference to FIGS. 2 to 4. FIG. 2 shows an example of a configuration of the information processing system 100 including an information processing apparatus 1 according to the example embodiment. As shown in FIG. 2, the information processing system 100 includes a doctor-side terminal 10 and a patient-side terminal 20.

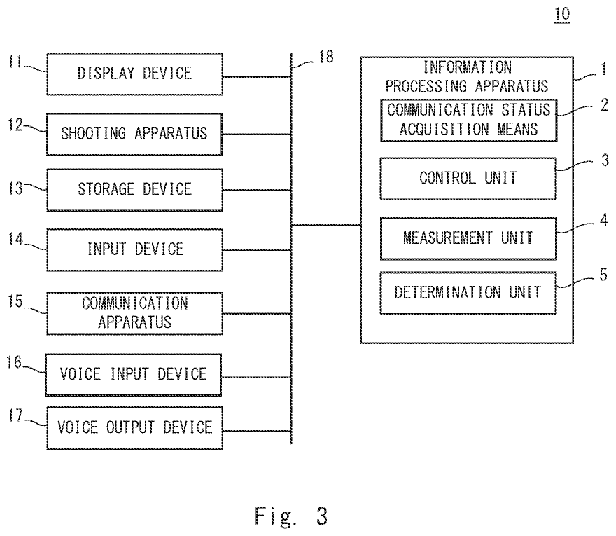

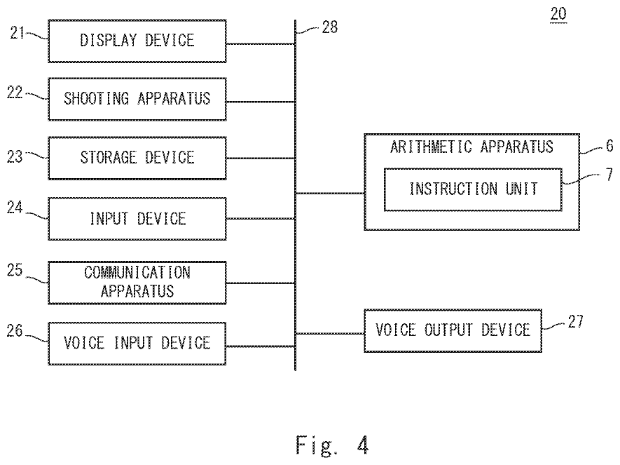

FIG. 3 is a block diagram showing an example of a configuration of the doctor-side terminal 10 shown in FIG. 2. FIG. 3 shows logical functional blocks implemented inside the information processing apparatus 1 as well as a hardware configuration of the doctor-side terminal 10. FIG. 4 is a block diagram showing an example of a configuration of the patient-side terminal 20 shown in FIG. 2. FIG. 4 shows logical functional blocks implemented inside an arithmetic apparatus 6 as well as a hardware configuration of the patient-side terminal 20.

The information processing system 100 supports remote medical care (online medical care or at-home medical care) in which a subject who receives a medical service receives various services from a medical service provider while staying in his/her house. Note that examples of medical service providers include a doctor, a nurse, a physical therapist, and a caregiver. Further, examples of remote medical care (or tele-medical care) include, in addition to medical practices in the narrow sense, such as a medical treatment and procedure performed by a doctor or the like, medical practices in the broad sense, such as a rehabilitation therapy performed by a physical therapist, health management and medicine-taking management performed by a caregiver. A medical service provider is referred to as a “doctor” to simplify the explanation.

Examples of subjects who receive medical services include a patient, a person who undergoes a medical examination (a health examinee), and a person who receives care (a care receiver). A subject who receives a medical service is referred to as a “patient” to simplify the explanation. Note that in the case of a DtoDtoP (Doctor to Doctor to Patient)-type remote medical care, a doctor may be present with the patient as the user using the patient-side terminal 20. Further, a nurse, a caregiver, or the like may be present with the patient to assist the medical examination of the patient. Examples of users of the patient-side terminal 20 include such medical stuff and the like. Further, examples of facilities that provide medical services include a hospital, a clinic, and a nursing home. A facility that provides medical services is referred to as a “hospital” to simplify the explanation.

The doctor-side terminal 10 and the patient-side terminal 20 are connected to each other through a network N, and perform two-way communication in real time. Note that the network N is a communication network such as the Internet or a private line. In the case of the Internet, a closed network is preferably formed by a VPN (Virtual Private Network) or the like.

The doctor-side terminal 10 is installed in a hospital, and the patient-side terminal 20 is installed in a house of a patient remotely located from the hospital. Each of the doctor-side terminal 10 and the patient-side terminal 20 may be a personal computer, a server machine, a tablet-type terminal and a smartphone capable of inputting/outputting data or the like through a touch panel, or the like. Each of the doctor-side terminal 10 and the patient-side terminal 20 may be a dedicated apparatus, or may be implemented by installing a dedicated application on a general-purpose information terminal. The doctor present in the hospital can, by using the doctor-side terminal 10, communicate with the patient who is in a place remotely located from the hospital and uses the patient-side terminal 20 in real time.

In such remote medical care using the information processing system 100, the doctor makes a diagnosis based on video images transmitted from the patient's home. The doctor-side terminal 10 can acquire biometric information of the patient from a video image(s) obtained by shooting (e.g., capturing a video image of) the patient. Biometric information is information about the living body of a person that can be measured (i.e., obtained) from a video image(s) of the person, and examples thereof include a heart rate, a blood pressure, and oxygen saturation.

The doctor-side terminal 10 can obtain such biometric information based on, for example, information indicating a change in the state of the body surface of the patient shown in a video image of the body surface. An example of information indicating a change in the state of a body surface is a change in the color of the body surface that occurs because of the flow of the blood in the patient. Each frame of video data obtained by shooting a patient includes (i.e., shows) a change in the color of the body surface of the patient. In the doctor-side terminal 10, the doctor or the like can measure a heart rate based on the changes in the color of the body surface of the patient.

Further, percutaneous arterial oxygen saturation (SpO₂) is a percentage representing how much oxygen is bound with hemoglobin in the blood. The intensity of light that is reflected from the body surface when light is applied to a body surface such as a face changes over time due to changes in the amount of hemoglobin in blood vessels. The doctor-side terminal 10 can estimate SpO₂ by analyzing a video image(s).

There are cases where when biometric information is measured based on such information that changes over time as described above, the communication status deteriorates. Note that the communication status is an indicator based on, for example, a communication bandwidth and/or throughput. Due to a disturbance of the video image or an increase in the communication delay caused by the deterioration of the communication situation, it becomes difficult to transmit the video image of the patient in real time.

For example, when the amount of image data transmitted from the patient-side terminal 20 to the doctor-side terminal 10 per unit time decreases, the accuracy of biometric information measured from this image data decreases. In the example embodiment, the quality of the medical service is prevented from deteriorating by providing necessary information to the doctor, the patient, and the user of the patient-side terminal 20 according to the communication status.

As shown in FIG. 3, the doctor-side terminal 10 includes an information processing apparatus 1, a display device 11, a shooting apparatus 12, a storage device 13, an input device 14, a communication apparatus 15, a voice input device 16, and a voice output device 17, all of which are connected to each other by a bus 18. The information processing apparatus 1 is an arithmetic processing apparatus such as a CPU (Central Processing Unit) or a GPU (Graphics Processing Unit).

The information processing apparatus 1 includes, as its logical functional blocks, a communication status acquisition unit 2, a control unit 3, a measurement unit 4, and a determination unit 5. The communication status acquisition unit 2, the control unit 3, the measurement unit 4, and the determination unit 5 correspond to communication status acquisition means, control means, measurement means, and determination means, respectively, specified in the claims.

The communication status acquisition unit 2 acquires a communication status which is one obtained (e.g., measured) when the doctor-side terminal 10 receives video data including a subject (i.e., containing an image of a subject) from the patient-side terminal 20 through the network N. Note that the video data including the patient may be one that is stored in a database (not shown) connected to the network. In this case, the communication status acquisition unit 2 can acquire the communication status which is one obtained (e.g., measured) when the video data is to be acquired from the database.

As described above, the communication status acquisition unit 2 can acquire, for example, a communication bandwidth obtained (or achieved) at that point in time or a communication delay occurring at that point in time as the communication status. To do this, a result of a self-diagnosis or the like of the patient-side terminal 20, to which the doctor-side terminal 10 is connected, can be used.

Further, the communication status acquisition unit 2 can predict, for example, fluctuations in the throughput for several tens of seconds ahead and acquire (i.e., use) them as the communication status. In an example, the communication status acquisition unit 2 can analyze fluctuations in the communication throughput for one minute in the past by artificial intelligence (AI), construct a prediction model in real time, and predict fluctuations in the communication throughput in the future.

Based on the communication status acquired by the communication status acquisition unit 2, the control unit 3 performs control so as to output instruction information for guiding at least one of the doctor, who is the user of the doctor-side terminal 10, the user of the patient-side terminal 20, and the patient to at least one of the doctor-side terminal 10 and the patient-side terminal 20. That is, the control unit 3 generates instruction information for guiding the doctor or the patient in regard to his/her actions in order to prevent the quality of the medical service from deteriorating.

The measurement unit 4 measures biometric information of the patient from video data including the patient (i.e., containing an image of the patient). Note that the measurement unit 4 may acquire relevant information related to the patient included in the video data and determines at least one item of biometric information to be measured based on one piece of relevant information or at least one combination of pieces of relevant information. The relevant information may include medical record information, medical inquiry information, ability information indicating the ability to perform activities of daily living such as ADL (Activities of Daily Living), information indicating records of prescription, nursing care, rehabilitation and the like, and information on a department of a hospital or the like in which the patient usually undergoes a medical examination. The above-described information may be stored and managed in an external management server.

Further, the measurement unit 4 may also determine that predetermined biometric information should not be regarded as an item to be measured based on one piece of relevant information at least one combination of pieces of relevant information. For example, types of biometric information to be measured are determined in advance for each department of a hospital or the like, and those that are not included in these types of biometric information determined in advance may be excluded from the items to be measured.

In the following description, an example in which the measurement unit 4 measures a heart rate as biometric information will be described. The measurement unit 4 detects a face area of the patient from the video data as a skin-color area representing a body surface, and measures a heart rate based on changes in the color of the face area over time. In the case where a heart rate is measured from a face area of video data of a patient as described above, for example, if a communication delay occurs and the time over which the color of the face area changes is changed, the reliability of the measured heart rate deteriorates.

Therefore, the determination unit 5 determines whether or not biometric information can be measured based on the communication status. The determination unit 5 can determine that, for example, when the communication delay is larger than a predetermined value, biometric information cannot be measured. The control unit 3 outputs information as to whether or not biometric information can be measured, obtained by the determination unit 5 to the doctor-side terminal 10. That is, the control unit 3 generates instruction information “Biological information cannot be measured due to the deterioration of the communication status.” to the doctor-side terminal 10. Regarding the start timing of measurement of biometric information by a doctor, it can be performed (i.e., started), by taking the communication situation into consideration, at a timing at which a high-quality video image of a patient can be taken. In this way, it is possible to prevent the doctor from making a wrong diagnosis which would otherwise be made due to unreliable biometric information.

Further, when the determination unit 5 determines that biometric information cannot be measured, the control unit 3 can also output, to the patient-side terminal 20, instruction information for guiding the patient so that biometric information can be measured. For example, when the communication bandwidth is narrow and the amount of data that can be transmitted per unit time is small, the control unit 3 can generate an instruction “Move the diseased part closer to the camera” to the patient-side terminal 20.

As the patient physically moves the diseased part closer to the camera, it is possible to obtain a video image of the patient having a resolution high enough to enable the doctor to make a correct diagnosis. By generating instruction information according to the communication status as described above, it is possible to guide the doctor and/or the patient in regard to his/her actions, and thereby to provide a higher quality medical service.

Further, for example, when a predicted value of the communication status is acquired, the determination unit 5 can also determine that biometric information will be able to be measured after a predetermined time (e.g., 10 seconds) indicated by the predicted value has elapsed. In this case, the control unit 3 can generate instruction information “In 10 seconds, the heart rate will be able to be measured. Please wait for a while” to the doctor-side terminal 10. In this way, the doctor can predict the waiting time.

Further, at this point, the control unit 3 can generate, for the patient-side terminal 20, an instruction “Measurement of the heart rate will start in 10 seconds. Please look at the camera squarely and then do not move your face”. In this way, the remote medical examination can be smoothly performed. Further, it is possible to request the patient to remain at a standstill at the timing at which the communication situation is satisfactory, and thereby to acquire a high-quality image of the patient.

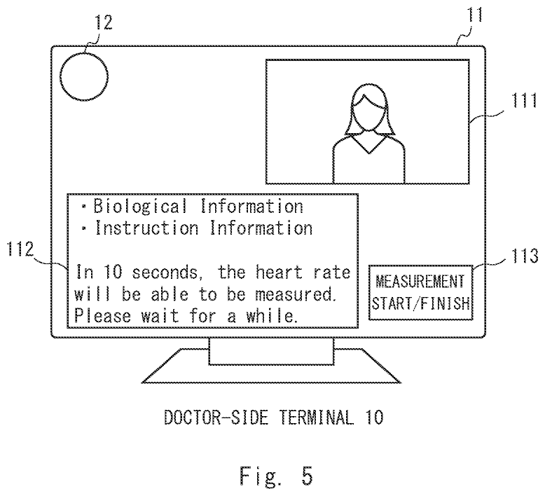

The display device 11 is an LCD (Liquid Crystal Display) or the like and displays various types of information. FIG. 5 is a diagram for explaining an example of contents displayed in the doctor-side terminal. As shown in FIG. 5, the display device 11 includes a video image display area 111, an information display area 112, and a measurement button 113.

In the video image display area 111, an image of a patient transmitted from the patient-side terminal 20 is displayed. In the information display area 112, biometric information measured from the video data of the patient is displayed. Further, in the information display area 112, instruction information for guiding the doctor in regard to his/her actions, generated according to the communication status is displayed.

The doctor can find, by referring to the instruction information, whether or not the measurement of biometric information can be performed. Further, when the biometric information cannot be measured, the doctor can know, for example, the time until the measurement can be performed. That is, in the example embodiment, the display device 11 serves as notification means for notifying the doctor, who is the user of the doctor-side terminal 10, of information as to whether or not biometric information can be measured. Note that the notification means is not limited to any particular means as long as it can notify the doctor of information as to whether or not biometric information can be measured through a voice, a notification sound pattern, a light emitting pattern, a vibration pattern, or the like.

Note that in the information display area 112, medical record information (e.g., a chronic disease, a medical history, examination information, and the like), inquiry information, and the like may be displayed. Further, the doctor-side terminal 10 may further include a voice analysis unit, and in the information display area 112, information related to contents of a conversation between the doctor and the patient, obtained through voice recognition may be displayed. For example, biometric information to be measured by the measurement unit 4 is determined from voice information “having a headache” spoken by the patient, and the display device 11 can display predetermined vital signs (a blood pressure, a heart rate, and the like).

Further, the information display area 112 may display candidate disease names according to the symptom by using the result of the voice recognition of the conversation between the doctor and the patient. Further, after the disease names are narrowed down according to the symptom, the information display area 112 may display recommendation information as to what kind of question should be asked in order to determine the disease name.

The shooting apparatus 12 is, for example, a video camera and can shoot (e.g., capture a video image of) the doctor who is using the doctor-side terminal 10. The shooting apparatus 12 shoots the doctor in a predetermined shooting range (shooting angle of view) at a predetermined shooting rate, and thereby generates moving image data composed of a plurality of time-series image data.

The storage device 13 is a non-volatile storage device, such as a hard disk drive (HDD), a solid-state drive (SSD), or a flash memory. The storage device 13 stores a program(s) for implementing the respective functions of the components provided in the information processing apparatus 1. When the information processing apparatus 1 executes this program, these functions of the components provided in the information processing apparatus 1 are implemented.

The storage device 13 may include a memory such as a RAM (Random Access Memory) and a ROM (Read Only Memory). When the information processing apparatus 1 executes the aforementioned program, the information processing apparatus 1 may execute the program after loading the program onto the memory, or may execute the program without loading it onto the memory. Further, the storage device 13 also has a function of storing information held by components provided in the information processing apparatus 1, such as information that the measurement unit 4 requires in order to measure biometric information.

The input device 14 is an apparatus that receives an operation input from the doctor, who is the user of the doctor-side terminal 10, such as a keyboard and mouse. The display device 11 and the input device 14 may be integrated and implemented as a touch panel. The doctor can, by using the input device 14, determine the start and end of the measurement of biometric information by clicking the measurement button 113.

Note that the information as to whether or not biometric information can be measured includes not only information indicating whether or not biometric information can be measured, but also information obtained by performing predetermined processing thereon. For example, the control unit 3 can output, based on the result of the determination by the determination unit 5, information as to whether or not measurement can be carried out in such a manner that the doctor can recognize, before he/she inputs data or the like to the input device 14, that the input device 14 does not accept an input. That is, the measurement button 113 displayed in the display device 11 is blanked (i.e., made to disappear). In this way, the doctor can find that receiving of an input is stopped before he/she inputs the start of measurement of biometric information.

Note that the control unit 3 may change the instruction information for the patient according to the input (the start and end of measurement of biometric information) to the input device 14. For example, when the doctor inputs the start of the measurement of a heart rate, the control unit 3 can output information (e.g., a notification) “Measurement of a heart rate will start now. Please look squarely at the camera.” to the patient-side terminal 20. Further, when the doctor inputs the end of the measurement of biometric information, the control unit 3 can output information “Measurement has been finished.” to the patient-side terminal 20. In this way, it is possible to enable the patient present in the remote place to know what the doctor will do, and thereby to support smooth communication between the doctor and the patient in online medical care.

The communication apparatus 15 transmits and receives data to and from the external patient-side terminal 20 through, for example, a wireless transmission line. For example, the communication apparatus 15 transmits video data including the doctor (i.e., containing an image of the doctor) to the patient-side terminal 20 and receives video data including the patient (i.e., containing an image of the patient) from the patient-side terminal 20. Further, when information necessary for the above-described various calculations is stored in an external management server, the communication apparatus 15 receives the information from the management server.

The voice input device 16 is, for example, a microphone and collects a voice uttered (or spoken) by the doctor as voice data. Video data is generated by combining the moving image data generated by the shooting apparatus 12 and the voice data collected by the voice input device 16.

The information display area 112 can also display information or the like by using the result of the voice recognition of the voice data of the doctor collected by the voice input device 16 and voice information contained in the video data including the patient. The voice output device 17 is, for example, a speaker and reproduces (or plays back) the voice contained in the video data of the patient transmitted from the patient-side terminal 20.

Next, the patient-side terminal 20 will be described. As shown in FIG. 4, the patient-side terminal 20 includes an arithmetic apparatus 6, a display device 21, a shooting apparatus 22, a storage device 23, an input device 24, a communication apparatus 25, a voice input device 26, and a voice output device 27, all of which are connected to each other through a bus 28.

Note that since the general functions of the display device 21, the shooting apparatus 22, the storage device 23, the input device 24, the communication apparatus 25, the voice input device 26, and the voice output device 27 are the same as those of the display device 11, the shooting apparatus 12, the storage device 13, the input device 14, the communication apparatus 15, the voice input device 16, and the voice output device 17, respectively, descriptions thereof will be omitted.

The arithmetic apparatus 6 is, for example, an arithmetic processing apparatus such as a CPU and a GPU. The arithmetic apparatus 6 includes an instruction unit 7 as a logical functional block. The instruction unit 7 outputs the instruction information for guiding the patient and/or the user of the patient-side terminal 20, transmitted from the doctor-side terminal 10. The instruction unit 7 can, for example, make the display device 21 (which will be described later) display the instruction information and make the voice output device 27 reproduce (or play back) the instruction information in the form of a voice.

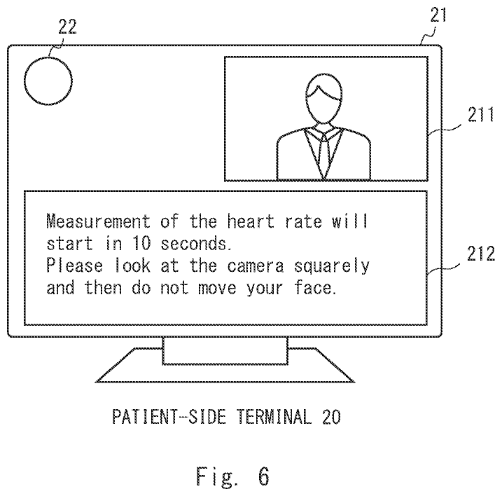

The display device 21 is an LCD or the like and displays various types of information. FIG. 6 is a diagram for explaining an example of contents displayed in the patient-side terminal. The display device 21 includes a video image display area 211 and an information display area 212. In the video image display area 211, video data including the doctor (i.e., containing an image of the doctor) transmitted from the doctor-side terminal 10 is displayed.

In the information display area 212, the instruction information for guiding the patient and/or the user of the patient-side terminal 20 is displayed. As described above, when it is determined that biometric information cannot be measured based on the communication status, the instruction unit 7 receives instruction information “Biometric information cannot be measured due to the deterioration of the communication status.”, and displays the received instruction information in the information display area 212. The instruction information for guiding the patient or the user of the doctor-side terminal 10, which is displayed in the information display area 212, includes, in addition to the above-described instruction to bring the face to a standstill, an instruction to move the face closer to the camera, an instruction to move the diseased part closer to the camera, and the like which are issued according to the biometric information to be measured.

Further, for example, when it is determined, based on relevant information, to measure a heart rate based on the video image of the patient, it is possible to display a notification or the like to notify the patient that a heart rate will be measured in the information display area 212 in advance.

Further, as described above, when a predicted value of the communication status is acquired and a heart rate is measured in 10 seconds, the instruction unit 7 receives instruction information “Measurement of the heart rate will start in 10 seconds. Please look at the camera squarely and then do not move your face.”, and displays the received instruction information in the information display area 212. In this way, the patient can know, from this notification, what is being measured, when the measurement is started, and what kind of actions (or movements) he/she should take, so that it is possible to guide the patient in regard to the adjustment of his/her own situation, and thereby to measure (i.e., obtain) reliable biometric information.

Note that the output of instruction information is not limited to the example in which the instruction unit 7 outputs the instruction information by displaying it in the display device 21. For example, the instruction unit 7 may make the voice output device 27 output instruction information in the form of a voice. Further, the instruction unit 7 can also output instruction information in the form of a notification sound or light, or a vibration pattern.

The shooting apparatus 22 is, for example, a video camera and can shoot (e.g., capture a video image of) the patient who is using the patient-side terminal 20. The shooting apparatus 22 shoots the patient in a predetermined shooting range (shooting angle of view) at a predetermined shooting rate, and thereby generates moving image data composed of a plurality of time-series image data.

The storage device 23 is a non-volatile storage device, such as an HDD, an SSD, or a flash memory. The storage device 23 stores a program(s) for implementing the function of the instruction unit 7 provided in the arithmetic apparatus 6. When the arithmetic apparatus 6 executes this program, the function of the instruction unit 7 is implemented. Further, similarly to the storage device 13, the storage device 23 may include a memory such as a RAM and a ROM. The storage device 13 also has a function of storing data and the like necessary for the instruction unit 7 to perform the process for outputting instruction information.

The input device 24 is an apparatus that receives an operation input from the patient, who is the user of the patient-side terminal 20, such as a keyboard and mouse. The display device 21 and the input device 24 may be integrated and implemented as a touch panel.

The communication apparatus 25 transmits and receives data to and from the external doctor-side terminal 10 through, for example, a wireless transmission line. For example, video data including the doctor (i.e., containing an image of the doctor), video data including the patient (i.e., containing an image of the patient), and instruction information are transmitted through the communication apparatus 25.

The voice input device 26 is, for example, a microphone and collects a voice uttered (or spoken) by the patient. Video data is generated by combining the moving-image data generated by the shooting apparatus 22 and the voice data collected by the voice input device 26. That is, this video data contains movement information and voice information of the patient. The voice output device 27 is, for example, a speaker and reproduces (or plays back) the voice contained in the video data of the doctor transmitted from the doctor-side terminal 10. Further, when instruction information for guiding the patient in regard to his/her actions is output in the form of a voice, the voice output device 27 can reproduce (or play back) this instruction information in the form of a voice.

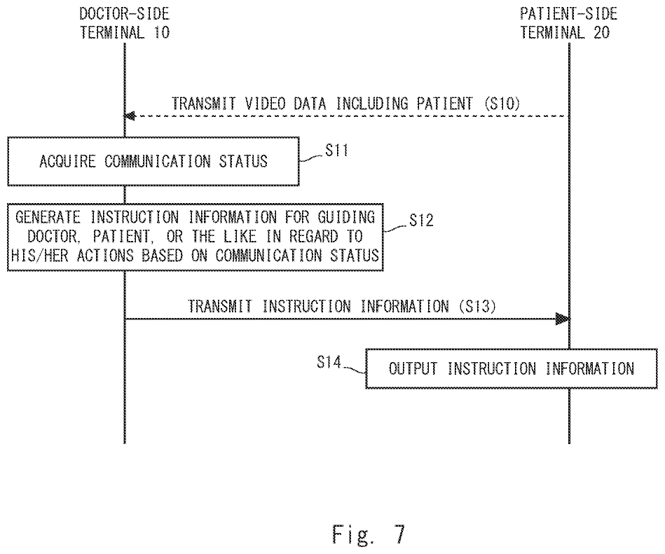

An information processing method according to an example embodiment will be described hereinafter with reference to FIG. 7. FIG. 7 is a sequence diagram for explaining an example of a flow of information processing according to an example embodiment. Firstly, the patient-side terminal 20 transmits video data including the patient to the doctor-side terminal 10 (S10). The communication status acquisition unit 2 of the doctor-side terminal 10 acquires the communication status which is one obtained (e.g., measured) when video data including the patient is transmitted (S11).

Then, the control unit 3 generates, based on the communication status, instruction information for guiding at least one of the doctor, the patient, and the user of the patient-side terminal 20 to at least one of the doctor-side terminal 10 and the patient-side terminal 20 (S12), and transmits the generated instruction information to the patient-side terminal 20 (S13). The instruction unit 7 of the patient-side terminal 20 acquires this instruction information and outputs the instruction information, for example, by displaying it in the display device 21 (S14).

As described above, according to the example embodiment, it is possible to provide instruction information for guiding the patient and/or the user of the patient-side terminal 20 in regard to his/her actions according to the communication status. In this way, the patient and/or the user of the patient-side terminal 20 is guided according to the instruction information, so that it is possible to improve measured biometric information and thereby to improve the medical service.

Note that although the information processing system 100 includes the information processing apparatus 1 in the example shown in FIG. 3, the present disclosure is not limited to this configuration. For example, the information processing apparatus 1 can be implemented as a server installed in a data center or the like. The image data including the patient (i.e., containing an image of the patient) obtained from the patient-side terminal 20 is transmitted to the server, and the server performs a process for generating the above-described instruction information.

Note that each of various functional blocks shown in the drawings, which perform various processes, can be implemented by hardware using a processor, a memory, and other circuits. Further, it is also possible to carry out the above-described processes by having a processor execute a program(s). Therefore, these functional blocks can be implemented in various ways by hardware alone, software alone, or a combination thereof, and are not limited to any of them.

The aforementioned programs may be stored in any of various types of non-transitory computer readable media and supplied to the computer. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Compact Disc-ROM), a CD-R, a CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line such as an electric wire and an optical fiber or a wireless communication line.

Although the present disclosure is described above with reference to example embodiments, the present disclosure is not limited to the above-described example embodiments. Various modifications that can be understood by those skilled in the art can be made to the configuration and details of the present disclosure within the scope of the disclosure.

Further, the whole or part of the example embodiments disclosed above can be described as, but not limited to, the following supplementary notes.

### (Supplementary Note 1)

An information processing system comprising:
communication status acquisition means for acquiring a communication status, the communication status being one when a first terminal receives a video image including a subject from a second terminal through a network; and
instruction means for outputting instruction information for guiding at least one of a user of the first terminal, a user of the second terminal, and the subject to at least one of the first and second terminals based on the acquired communication status.

### (Supplementary Note 2)

The information processing system described in Supplementary note 1, further comprising:
measuring means for measuring biometric information of the subject; and
determination means for determining whether or not the biometric information can be measured based on the communication status, wherein
the instruction means outputs information as to whether or not the biometric information can be measured, determined by the determination means to the first terminal.

### (Supplementary Note 3)

The information processing system described in Supplementary note 2, wherein when the determination means determines that the biometric information cannot be measured, the instruction means outputs, to the second terminal, instruction information for guiding the subject so that the biometric information can be measured.

### (Supplementary Note 4)

The information processing system described in Supplementary note 2 or 3, further comprising receiving means for receiving an input for starting measurement of the biometric information, wherein
the instruction means outputs, based on a result of the determination by the determination means, the information as to whether or not the measurement can be carried out so that it can be known, before the receiving means receives an input, that the receiving means does not accept an input.

### (Supplementary Note 5)

The information processing system described in any one of Supplementary notes 1 to 4, wherein
the first terminal comprises first display means for displaying a video image of the subject and displaying the instruction information from the instruction means, and
the second terminal comprises second display means for displaying the instruction information from the instruction means.

### (Supplementary Note 6)

An information processing apparatus comprising:
communication status acquisition means for acquiring a communication status, the communication status being one when a first terminal receives a video image including a subject from a second terminal through a network; and
control means for performing control so as to output instruction information for guiding at least one of a user of the first terminal, a user of the second terminal, and the subject to at least one of the first and second terminals based on the acquired communication status.

### (Supplementary Note 7)

The information processing apparatus described in Supplementary note 6, further comprising:
measuring means for measuring biometric information of the subject; and
determination means for determining whether or not the biometric information can be measured based on the communication status, wherein
the control means performs control so as to output information as to whether or not the biometric information can be measured, determined by the determination means to the first terminal.

### (Supplementary Note 8)

The information processing apparatus described in Supplementary note 7, wherein when the determination means determines that the biometric information cannot be measured, the control means performs control so as to output, to the second terminal, instruction information for guiding the subject so that the biometric information can be measured.

### (Supplementary Note 9)

The information processing apparatus described in Supplementary note 7 or 8, further comprising receiving means for receiving an input for starting measurement of the biometric information, wherein
the control means performs control so as to output, based on a result of the determination by the determination means, the information as to whether or not the measurement can be carried out so that it can be known, before the receiving means receives an input, that the receiving means does not accept an input.

### (Supplementary Note 10)

The information processing apparatus described in any one of Supplementary notes 6 to 9, wherein the control means:
makes first display means included in the first terminal display a video image of the subject and display the instruction information; and
makes second display means included in the second terminal display the instruction information.

### (Supplementary Note 11)

An information processing method comprising:
acquiring a communication status, the communication status being one when a first terminal receives a video image including a subject from a second terminal through a network; and
outputting instruction information for guiding at least one of a user of the first terminal, a user of the second terminal, and the subject to at least one of the first and second terminals based on the acquired communication status.

### (Supplementary Note 12)

The information processing method described in Supplementary note 11, further comprising:
measuring biometric information of the subject;
determining whether or not the biometric information can be measured based on the communication; and
outputting information as to whether or not the biometric information can be measured to the first terminal.

### (Supplementary Note 13)

The information processing method described in Supplementary note 12, wherein when it is determined that the biometric information cannot be measured, instruction information for guiding the subject is output to the second terminal so that the biometric information can be measured.

### (Supplementary Note 14)

The information processing method described in Supplementary note 12 or 13, wherein, based on a result of the determination as to whether or not the measurement can be carried out, the information as to whether or not the biometric information can be measured is output so that it can be known that, before the receiving means receiving an input for starting measurement of the biometric information receives an input, that the receiving means does not accept an input.

### (Supplementary Note 15)

The information processing method described in any one of Supplementary notes 11 to 14, further comprising:
making first display means included in the first terminal display a video image of the subject and display the instruction information; and
making second display means included in the second terminal display the instruction information.

### (Supplementary Note 16)

A non-transitory computer readable medium storing a program for causing a computer to perform processes including:
acquiring a communication status, the communication status being one when a first terminal receives a video image including a subject from a second terminal through a network; and
outputting instruction information for guiding at least one of a user of the first terminal, a user of the second terminal, and the subject to at least one of the first and second terminals based on the acquired communication status.

### (Supplementary Note 17)

The non-transitory computer readable medium storing the program described in Supplementary note 16, wherein the program further causes the computer to:
measure biometric information of the subject;
determine whether or not the biometric information can be measured based on the communication status; and
output information as to whether or not the biometric information can be measured to the first terminal.

### (Supplementary Note 18)

The non-transitory computer readable medium storing the program described in Supplementary note 17, wherein when it is determined that the biometric information cannot be measured, instruction information for guiding the subject is output to the second terminal so that the biometric information can be measured.

### (Supplementary Note 19)

The non-transitory computer readable medium storing the program described in Supplementary note 17 or 18, wherein the program further causes the computer to output, based on a result of the determination as to whether or not the measurement can be carried out, the information as to whether or not the measurement can be carried out so that it can be known that, before the receiving means receiving an input for starting measurement of the biometric information receives an input, that the receiving means does not accept an input.

### (Supplementary Note 20)

The non-transitory computer readable medium storing the program described in any one of Supplementary notes 16 to 19, wherein the program further causes the computer to:
make first display means included in the first terminal display a video image of the subject and display the instruction information; and
make second display means included in the second terminal display the instruction information.

### REFERENCE SIGNS LIST

1 INFORMATION PROCESSING APPARATUS
2 COMMUNICATION STATUS ACQUISITION UNIT
3 CONTROL UNIT
4 MEASUREMENT UNIT
5 DETERMINATION UNIT
6 ARITHMETIC APPARATUS
7 INSTRUCTION UNIT
10 DOCTOR-SIDE TERMINAL
11 DISPLAY DEVICE
12 SHOOTING APPARATUS
13 STORAGE DEVICE
14 INPUT DEVICE
15 COMMUNICATION APPARATUS
16 VOICE INPUT DEVICE
17 VOICE OUTPUT DEVICE
18 BUS
20 PATIENT-SIDE TERMINAL
21 DISPLAY DEVICE
22 SHOOTING APPARATUS
23 STORAGE DEVICE
24 INPUT DEVICE
25 COMMUNICATION APPARATUS
26 VOICE INPUT DEVICE
27 VOICE OUTPUT DEVICE
28 BUS
100 INFORMATION PROCESSING SYSTEM
101 COMMUNICATION STATUS ACQUISITION MEANS
102 INSTRUCTION MEANS
111 VIDEO IMAGE DISPLAY AREA
112 INFORMATION DISPLAY AREA
113 MEASUREMENT BUTTON
211 VIDEO IMAGE DISPLAY AREA
212 INFORMATION DISPLAY AREA
N NETWORK

What is claimed is:

1. An information processing system comprising:
at least one memory storing instructions, and
at least one processor configured to execute the instructions to;
acquire a communication status, the communication status being one when a first terminal receives a video image including a subject from a second terminal through a network; and
output instruction information for guiding at least one of a user of the first terminal, a user of the second terminal, and the subject to at least one of the first and second terminals based on the acquired communication status.

2. The information processing system according to claim 1, wherein at least one processor is further configured to execute the instructions to:
measure biometric information of the subject; and
determine whether or not the biometric information can be measured based on the communication status, wherein
information as to whether or not the biometric information can be measured is output to the first terminal.

3. The information processing system according to claim 2, wherein when it is determined that the biometric information cannot be measured, instruction information for guiding the subject is output to the second terminal so that the biometric information can be measured.

4. The information processing system according to claim 2, wherein at least one processor is further configured to execute the instructions to:
receive an input for starting measurement of the biometric information, wherein
based on a result of the determination, the information as to whether or not the measurement can be carried out is output so that it can be known, before the receiving an input, that the receiving of an input is not accepted.

5. The information processing system according to claim 1, wherein
the first terminal comprises first display for displaying a video image of the subject and displaying the instruction information, and
the second terminal comprises second display for displaying the instruction information.

6-10. (canceled)

11. An information processing method, wherein a computer
acquires a communication status, the communication status being one when a first terminal receives a video image including a subject from a second terminal through a network; and
outputs instruction information for guiding at least one of a user of the first terminal, a user of the second terminal, and the subject to at least one of the first and second terminals based on the acquired communication status.

12. The information processing method according to claim 11, wherein the computer
measures biometric information of the subject;
determines whether or not the biometric information can be measured based on the communication; and
outputs information as to whether or not the biometric information can be measured to the first terminal.

13. The information processing method according to claim 12, wherein when it is determined that the biometric information cannot be measured, instruction information for guiding the subject is output to the second terminal so that the biometric information can be measured.

14. The information processing method according to claim 12, wherein, based on a result of the determination as to whether or not the measurement can be carried out, the information as to whether or not the biometric information can be measured is output so that it can be known that, before the receiving means receiving an input for starting measurement of the biometric information receives an input, that the receiving means does not accept an input.

15. The information processing method according to claim 11, wherein the computer
makes first display included in the first terminal display a video image of the subject and display the instruction information; and
makes second display included in the second terminal display the instruction information.

16. A non-transitory computer readable medium storing a program for causing a computer to perform processes including:
acquiring a communication status, the communication status being one when a first terminal receives a video image including a subject from a second terminal through a network; and
outputting instruction information for guiding at least one of a user of the first terminal, a user of the second terminal, and the subject to at least one of the first and second terminals based on the acquired communication status.

17. The non-transitory computer readable medium storing the program according to claim 16, wherein the program further causes the computer to:
measure biometric information of the subject;
determine whether or not the biometric information can be measured based on the communication status; and
output information as to whether or not the biometric information can be measured to the first terminal.

18. The non-transitory computer readable medium storing the program according to claim 17, wherein when it is determined that the biometric information cannot be measured, instruction information for guiding the subject is output to the second terminal so that the biometric information can be measured.

19. The non-transitory computer readable medium storing the program according to claim 17, wherein the program further causes the computer to output, based on a result of the determination as to whether or not the measurement can be carried out, the information as to whether or not the measurement can be carried out so that it can be known that, before a receiving means receiving an input for starting measurement of the biometric information receives an input, that the receiving means does not accept an input.

20. The non-transitory computer readable medium storing the program according to claim 16, wherein the program further causes the computer to:
make first display included in the first terminal display a video image of the subject and display the instruction information; and
make second display included in the second terminal display the instruction information.
